(19) United States
(12) Patent Application Publication
(10) Pub. No.: US 2008/0243178 A1
(43) Pub. Date: Oct. 2, 2008

(54) Surgical instrument particularly useful as tweezers for grasping and holding objects of different thicknesses

(75) Inventors: Ran Oren, Kibbutz Gaaton (IL); Eran Zakai, Moshav Yodfat (IL); Dan Moor, Kibbutz Gaaton (IL)

Correspondence Address: Martin D. Moynihan;PRTSI, Inc., P.O. Box 16446, Arlington, VA, 22215, US

(73) Assignee: T.A.G. Medical Products a Limited Partnership, Doar-Na Oshrat (IL)

(21) Appl. No.: 11/729,942

(22) Filed: Mar. 30, 2007

(51) Int. Cl. A61B 17/28

(52) U.S. Cl. 606/210

(57) ABSTRACT

A surgical instrument useful as a tweezers for grasping and holding objects of different thicknesses includes: an elongated arm having a distal end defining a fixed jaw; a pivotal jaw having a distal end movable towards and away from the fixed jaw to a fully open position, a fully closed position, and at least one intermediate position, with respect to the fixed jaw; and a locking member carried by the pivotal jaw and manually movable into engagement with a locking surface carried by the elongated arm, when the pivotal jaw is in its fully-closed position or in any intermediate position with respect to the fixed, to lock the pivotal jaw in the fully-closed or intermediate position with respect to the fixed jaw.

### FIELD AND BACKGROUND OF THE INVENTION

The present invention relates to surgical instruments, and particularly to instruments useful as tweezers or forceps during a surgical procedure for grasping and holding objects of different thicknesses, such as various organs or tissues of a patient's body or other medical devices such as sutures and suturing needles.

The foregoing type of instruments typically consist of two opposed jaws at the distal end of the instrument, at least one of them being movable in relation to the other, and means for actuating the movable jaw. Conventional instruments, of generally two types in many sizes, are available for specific applications.

The first type is of a scissors or pliers-like construction. It consists of two elongated members each having a distal end (with respect to the user) and a proximal end. The distal ends are formed as jaws suitable for the intended application, while the proximal ends serve as handles for manipulating the jaws. The two members are pivotally connected to each other between their proximal and distal ends, such that moving the handles towards each other will cause the jaws to close.

The second type is of a tweezers-like construction. This type includes two elongated members having the jaws formed at their distal ends connected together at their proximal ends. In clamping instruments, the arms are biased to an open position, and are closed by pressing them together. In some tweezers intended for dissecting, the arms are biased to a normally closed position and can be actuated by a wedge-shaped element forced between them for separation.

In many applications it is desirable to lock the jaws of the instrument on an objects of different thicknesses, or in any position along the range of motion of the jaws. In scissors-type instruments this is achieved by placing a releasable ratchet between the handles. The ratchet may consist of a rod provided with saw-teeth serrations. The rod is pivotally mounted to one of the arms and is biased by a spring to engage a detent mounted in the opening arm. Actuating a lever formed integral with the serrated rod will release the mechanism. An alternative ratchet consists of a surface with saw teeth provided on one handle and with a detent on the other. The detent remains normally in engagement, and the handles must be forced apart to release it. Yet another ratchet like locking mechanism is disclosed in U.S. Pat. No. 5,876,420 to Noll, et al.

Locking mechanisms are rarely used on tweezers-like instruments. One such mechanism is disclosed in U.S. Pat. No. 5,405,353 to Randall. However, the locking of that construction allows only for a fully closed or a fully open position; therefore it is unsuitable for clamping objects of varying thicknesses.

### OBJECTS AND BRIEF SUMMARY OF THE PRESENT INVENTION

An object of the present invention is to provide a surgical instrument of the tweezers-type which may be locked, not only in a fully-closed position or in a fully-open position, but also in any intermediate position therebetween, to thereby enable the instrument to securely clamp tissues or other objects of different thicknesses. Another object of the present invention is to provide such a surgical instrument which is particularly useful as a finger-mountable instrument for both open surgery and for endoscopic surgery.

According a broad aspect of the present invention, there is provided a surgical instrument useful as a tweezers for grasping and holding objects of different thicknesses, comprising: an elongated arm having a proximal end, and a distal end defining a fixed jaw; a pivotal jaw having a distal end pivotal towards and away from the fixed jaw to a fully open position, a fully closed position, and at least one intermediate position, with respect to the fixed jaw; and a locking member carried by the pivotal jaw and manually movable with respect thereto into engagement with a locking surface carried by said elongated arm when the pivotal jaw is in its fully-closed position and in any intermediate position with respect to the fixed jaw, to lock the pivotal jaw in the fully-closed or intermediate position with respect to the fixed jaw.

In the described preferred embodiment, the surgical instrument further comprises a spring normally urging the pivotal jaw to its fully open position. Also, the elongated arm carries a short arm extending between the elongated arm and carrying the locking surface engageable by the locking member. The short arm is formed with spaced bifurcations each carrying a locking surface. The locking member includes a slidable button slidably mounted on the pivotal jaw, and a locking element extending from the slidable button into the space between the bifurcations and engageable with the locking surfaces.

According to still further features in the described preferred embodiment, the locking surfaces of the bifurcations are teeth formed in in-turned ends of the bifurcations effective to lock the pivotal jaw in any one of a plurality of intermediate positions. The locking element includes a stem fixed to the slidable button and formed with an out-turned flange receivable between the teeth. In the described preferred embodiments the stem is of a cylindrical configuration, and the out-turned flange is formed at the end thereof facing the fixed jaw. According to still further features in the described preferred embodiment, the teeth have inclined faces facing the pivotal jaw, and perpendicular faces facing the fixed jaw.

The invention is described particularly with respect to a finger-mountable surgical instrument, including a sleeve dimensioned to be received in the outer end of a finger of a user (e.g., surgeon). It will be appreciated, however, that the invention could also be used in conventional hand-grippable surgical instruments.

Further features and advantages of the invention will be apparent from the description below.

### BRIEF DESCRIPTION OF THE DRAWINGS

The invention is herein described, by way of example only, with reference to the accompanying drawings, wherein:

FIG. 1 illustrates one form of finger-mountable surgical instrument constructed in accordance with the present invention;

FIG. 2 is a side view of the surgical instrument of FIG. 1;

FIGS. 3 and 4 are end views, from opposite ends, of the surgical instrument of FIGS. 1 and 2; and

FIG. 5 illustrates a modification in the surgical instrument and showing the two jaws locked in their fully closed position.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF A PREFERRED EMBODIMENT

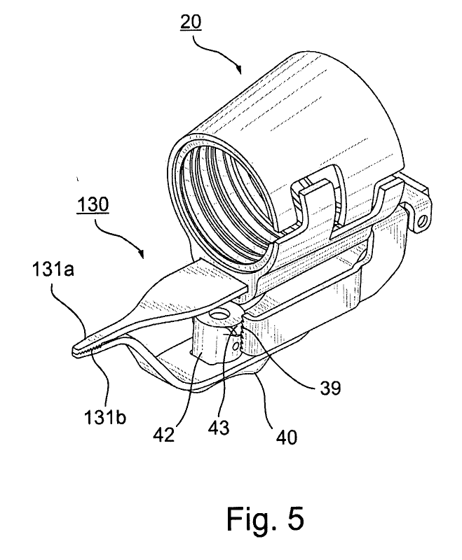

The preferred embodiment of the invention illustrated in the drawings is a finger-mountable surgical instrument useful as a tweezers for grasping objects of different thicknesses. It includes a sleeve, generally designated 20, dimensioned to be received on the outer end of the finger of a user (e.g., a surgeon), and a pair of tweezers, generally designated 30, having two arms defining two jaws which may be selectively locked in a fully-closed position, as illustrated in FIG. 5, or in any intermediate open position. The invention of the present application involves the construction of the surgical instrument 30 which enables its two jaws to be locked in any desired position, and while the drawings illustrate the instrument as being of the finger-mountable type, it will be appreciated that the invention could also be used in a conventional hand-graspable type of surgical instrument.

For purposes of example, the finger-mountable surgical instrument illustrated in the drawings utilizes a sleeve 20 of the construction in concurrently-filed patent application Ser. No.: ______, assigned to the same assignee as the present application. Such a sleeve has an open proximal end 21 (with respect to the surgeon) for application to the outer end of the surgeon's finger, and an open distal end 22 which exposes the outer tip of the surgeon's finger when mounted thereon. Sleeve 20 is of a tapered construction, having an inner diameter and an outer diameter decreasing from its proximal end 21 to its distal end 22, which facilitates the application of the sleeve to the surgeon's finger.

Sleeve 20 is split along its longitudinal axis so as to define two opposed edges 23, 24, confronting each other. The two edges 23, 24, includes interengaging ribs (23a, 24a) and recesses (23b, 24b). The inner surface of sleeve is formed with circumferentially-extending ribs, to assure gripping of the surgeon's gloved finger when the sleeve is mounted thereon.

Sleeve 20 is made of an elastic, preferably plastic, material such that the longitudinal—split permits its diameter to be increased in order to accommodate fingers of different thicknesses. The interengaging ribs and recesses not only better assure that the sleeve will firmly engage the entire outer surface of the surgeon's gloved finger when mounted thereon, but also facilitate the application of the sleeve to the finger of the surgeon without “pinching” the finger.

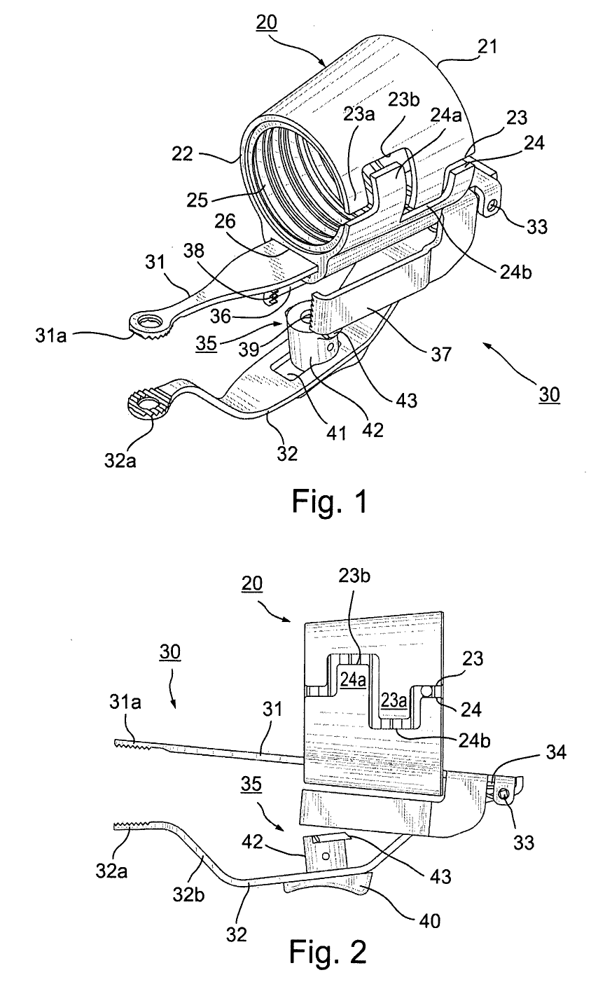

One side of sleeve 20 is integrally formed with a socket 26, defining a through-going passageway, for receiving the surgical instrument to be finger-mounted by the sleeve. In this case, the surgical instrument is the tweezers 30, having one arm 31 received within socket 26, and a second arm 32 pivotally mounted to arm 31 by pivot pin 33. The distal end of arm 31 defines a fixed jaw 31a, and the distal end of arm 32 defines a pivotal jaw 32a movable towards and away from the fixed jaw 31a. As will be described more particularly below, pivotal jaw 32a is movable to a fully open position with respect to fixed jaw 31a (as shown in FIGS. 1 and 2), to a fully closed position with respect to the fixed jaw (as shown in FIG. 5), or to any intermediate position with respect to fixed jaw. Movable jaw 32a is normally biased to its fully-open position by a piano spring 34 (FIGS. 2 and 4).

The surgical instrument further includes a locking member, generally designated 35, carried by pivotal jaw 32a and manually slidable with respect thereto into engagement with a locking surface carried by fixed jaw 31a. As will be described more particularly below, when the pivotal jaw is in its fully-closed position, or in any intermediate position, locking member 35 is manually slidable on pivotal arm 32 to lock the pivotal jaw in such fully-closed or intermediate position with respect to the fixed jaw 31a.

As shown particularly in FIG. 1, fixed arm 31 of tweezers 30 is integrally formed with a short arm having two spaced bifurcations, 36, 37, in the space between the two arms 31, 32 defining the two jaws 31a, 31b. The two bifurcations 36, 37 are formed, at their outer tips, with in-turned ends each having serrations or teeth, 38, 39. Teeth 38, 39 constitute locking surfaces which cooperate with locking member 35 to lock the two jaws 31a, 31b, in their completely closed condition, or in any intermediate partially open condition, as will be described more particularly below.

Locking member 35 includes a button 40 slidable in a slot 41 formed in arm 32 defining the movable jaw 32a, and a locking element 42 in the form of a stem projecting through slot 41 and between the two bifurcations, 36, 37. Stem 42 is of cylindrical configuration and includes, at its outer end, a pair of projections or flanges 43 selectively receivable between teeth 38 and 39 of bifurcations 36 and 37 for locking the pivotal jaw 32a in any selected position with respect to the fixed jaw 31a.

Slidable button 40 preferably has an outer concave configuration, to accommodate the user's thumb, when the instrument is finger-mounted (or hand-grasped), to allow the user first to depress the button, and thereby to move pivotal jaw 32a to a desired position with respect to fixed jaw 31a. The user may then slide the button in the forward (distal) direction to cause flanges 43 of stem 42 to be received between the teeth 38, 39 of the two bifurcations 36, 37, with which the flanges are aligned in the selected position in which the pivotal jaw 32a is to be fixed with respect to the fixed jaw 31a.

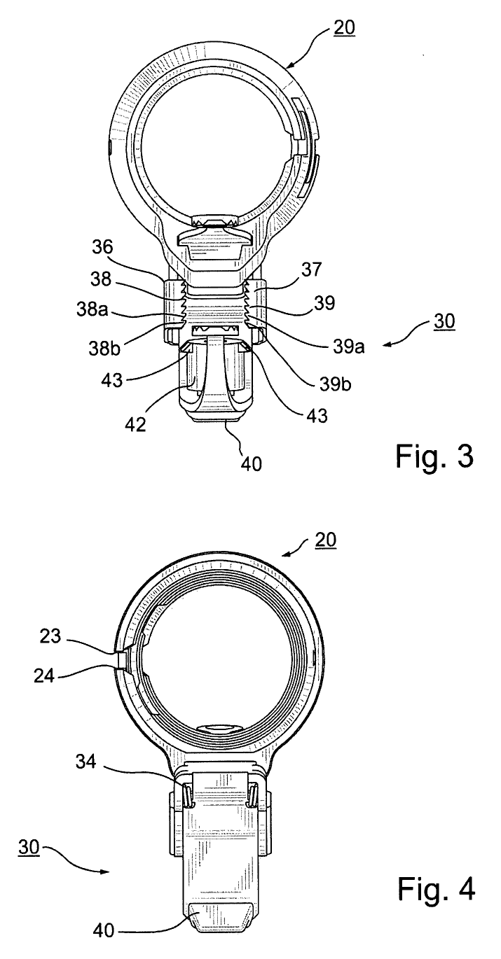

As shown in FIG. 3, teeth 38 and 39 include inclined faces 38a, 39a facing the pivotal arm 32 defining the pivotal jaw 32a, and perpendicular faces 38b, 39b facing the fixed jaw 31a. Thus, when button 40 is pressed, it aligns flanges 43 of stem 42 with teeth 38, 39, according to the desired intermediate position of pivotal jaw 32a with respect to fixed jaw 31a, such that sliding this button in the forward direction will lock the pivotal jaw in that position. The user, by further pressing button 40, may if desired decrease the spacing by further moving pivotal jaw 32a towards fixed jaw 31a, this being permitted by the slanted faces 38a, 39a of the locking teeth 38, 39. However, the pivotal jaw is locked from opening further, or from moving to its fully open position, by the perpendicular faces 38b, 39b, of the locking teeth 38, 39.

The manner of using the surgical instrument illustrated in the drawings will be apparent from the above description.

Thus, if the instrument is included in a finger-mountable instrument, as illustrated in FIG. 1, the user (surgeon) would first insert his index finger through sleeve 20, such that the two jaws 31a, 32a, project forwardly of the instrument, and finger button 40 is disposed below the sleeve for convenient engagement by the thumb of the surgeon. Spring 34 normally urges pivotal jaw 32a to its fully open position with respect to fixed jaw 31a, as shown in FIGS. 1 and 2.

Whenever the surgeon wishes to grasp an object, e.g., tissue, an organ, a suture needle or the like, the surgeon presses finger button 40 inwardly (e.g. towards the fixed arm 31), to grasp the object between the two jaws, or to bring the pivotal jaw 32a to the desired spacing distance with respect to the fixed jaw 31a. The surgeon then slides finger button 40 forwardly (i.e. towards pivotal jaw 32a) to locate the out-turned flanges 42 of stem 41 between the teeth 38, 39 formed in the in-turned bifurcations 36, 37, for locking the pivotal jaw 32a in the desired position with respect to the fixed jaw 31a. Because of the configuration of the teeth 38, 39 as shown in FIG. 3, the surgeon can decrease the spacing between the two jaws 31a, 32a, if desired, by merely pressing further on finger button 40. However, once the desired spacing is obtained, the perpendicular faces 38b, 39b, of the teeth lock the pivotal jaw 32a in the respective position with respect to fixed jaw 31a, and prevent the outward movement of the pivotal jaw with respect to the fixed jaw.

As seen particularly in FIGS. 1 and 2, the distal end of pivotal arm 32 defining pivotal jaw 32a is bent towards the fixed arm 31 defining the fixed jaw 31a, as shown at 32b, such that the pivotal jaw 32a is approximately parallel to the fixed jaw 31a in all pivotal positions of the pivotal jaw.

As seen particularly in FIG. 1, the two jaws 31a, 32a, used for grasping tissue or other objects are serrated and are of an annular configuration so as to present a large surface-engaging area for contact with the tissue or other object to be grasped.

FIG. 5 illustrates a modification in the construction of the tweezers, therein designated 130, in which the two jaws 131a, 131b are serrated but of a linear configuration to enable better grasping small objects, such as sutures or suture needles. In all other respects, the finger-mountable tweezers illustrated in FIG. 5 is of substantially the same construction as described above with respect to FIGS. 1-4, and therefore the same reference numerals have been used to identify corresponding parts.

While the invention has been described with respect to a preferred embodiment, it will be appreciated that this is set forth merely for purposes for example, and that many variations can be made. For example, and as indicated above, the invention could also be incorporated in a conventional hand-graspable surgical instrument. Many other variations, modifications and applications of the invention will be apparent.

What is claimed is:

1. A surgical instrument useful as a tweezers for grasping and holding objects of different thicknesses, comprising:
an elongated arm having a proximal end, and a distal end defining a fixed jaw;
a pivotal jaw having a distal end pivotal towards and away from said fixed jaw to a fully open position, a fully closed position, and at least one intermediate position, with respect to said fixed jaw;
and a locking member carried by said pivotal jaw and manually movable with respect thereto into engagement with a locking surface carried by said elongated arm, when the pivotal jaw is in its fully-closed position and in any intermediate position with respect to said fixed jaw, to lock said pivotal jaw in said fully-closed or intermediate position with respect to said fixed jaw.

2. The surgical instrument according to claim 1, further comprising a spring manually urging said pivotal jaw to its fully open position.

3. The surgical instrument according to claim 1, wherein said elongated arm includes a short arm extending between said elongated arm and said pivotal jaw and carrying said locking surface engageable by said locking member.

4. The surgical instrument according to claim 3, wherein said short arm is formed with spaced bifurcations each carrying a said locking surface.

5. The surgical instrument according to claim 4, wherein said locking member includes a button slidably mounted on said pivotal jaw, and a locking element extending from said slidable button into the space between said bifurcations and engageable with the locking surfaces thereof.

6. The surgical instrument according to claim 5, wherein said pivotal jaw is pivotally mounted between said bifurcations at the proximal end of said elongated arm.

7. The surgical instrument according to claim 5, wherein said locking surfaces of said bifurcations are teeth formed in in-turned ends of said bifurcations effective to lock said pivotal jaw in any one of a plurality of intermediate positions.

8. The surgical instrument according to claim 7, wherein said locking element includes a stem fixed to said slidable button and formed with an out-turned flange receivable between said teeth.

9. The surgical instrument according to claim 6, wherein said stem is of a cylindrical configuration.

10. The surgical instrument according to claim 7, wherein said teeth have inclined faces facing said pivotal jaw, and perpendicular faces facing said fixed jaw.

11. The surgical instrument according to claim 1, wherein said distal end of the pivotal jaw is bent towards said fixed jaw so as to be approximately parallel thereto in all pivotal positions of the pivotal jaw.

12. The surgical instrument according to claim 1, wherein said proximal end of elongated arm is fixed to a sleeve dimensioned to be received on the outer end of a finger of a user.

13. A surgical instrument useful as a tweezers for grasping objects, comprising:
an elongated arm having a proximal end and a distal end defining a fixed jaw;
a pivotal jaw pivotally mounted to the proximal end of said elongated arm and having a distal end movable towards and away from said fixed jaw to a fully open position, a fully closed position, and at least one intermediate position, with respect to said fixed jaw;
a spring normally urging said pivotal jaw to its fully open position;
and a locking member carried by a button slidable on said pivotal jaw and manually slidable with respect thereto into engagement with a locking surface fixed to said elongated arm when the pivotal jaw is in its fully-closed position or in any intermediate position with respect to said fixed jaw, to lock said pivotal jaw in said fully-closed or intermediate position with respect to said fixed jaw.

14. The surgical instrument according to claim 1, wherein said elongated arm is fixed to a short arm extending between said elongated arm and said pivotal jaw and parallel to said elongated arm, said short arm carrying said locking surface engageable by said locking member.

15. The surgical instrument according to claim 14, wherein said short arm is formed with spaced bifurcations each carrying a said locking surface; and said locking member includes a locking element extending from said slidable button into the space between said bifurcations and engageable with the locking surfaces thereof.

16. The surgical instrument according to claim 15, wherein said locking element includes a stem fixed to said slidable button and formed with an out-turned flange receivable between said teeth.

17. The surgical instrument according to claim 16, wherein said stem is of a cylindrical configuration, and said out-turned flange is formed at the end thereof proximal to said fixed jaw.

18. The surgical instrument according to claim 15, wherein said pivotal jaw is pivotally mounted between said bifurcations, and said locking surfaces of said bifurcations are teeth formed in in-turned ends of said bifurcations effective to lock said pivotal jaw in any one of a plurality of intermediate positions.

19. The surgical instrument according to claim 5, wherein said teeth have inclined faces facing said pivotal jaw, and perpendicular faces facing said fixed jaw.

20. The surgical instrument according to claim 13, wherein the proximal end of said elongated arm is fixed to a sleeve dimensioned to be received on the outer end of a finger of a user.
